(19) United States
(12) Patent Application Publication
(10) Pub. No.: US 2008/0053436 A1
(43) Pub. Date: Mar. 6, 2008

(54) AEROSOL CREATED BY DIRECTED FLOW OF FLUIDS AND DEVICES AND METHODS FOR PRODUCING SAME

(75) Inventors: Joan Rosell, Castro Valley, CA (US); Alfonso Ganan-Calvo, Sevilla (ES)

Correspondence Address: BOZICEVIC, FIELD & FRANCIS LLP, 1900 UNIVERSITY AVENUE, SUITE 200, EAST PALO ALTO, CA, 94303, US

(73) Assignees: Aradigm Corporation; Universidad de Sevilla

(21) Appl. No.: 11/929,642

(22) Filed: Oct. 30, 2007

Related U.S. Application Data
(63) Continuation of application No. 11/615,732, filed on Dec. 22, 2006.
Application No. 11/615,732 is a continuation of application No. 10/649,376, filed on Aug. 26, 2003.
Application No. 10/649,376 is a continuation-in-part of application No. 09/591,365, filed on Jun. 9, 2000, now abandoned.
(60) Provisional application No. 60/138,698, filed on Jun. 11, 1999.

(51) Int. Cl. A61M 16/00; A61M 11/06; B05B 7/00

(52) U.S. Cl. 128/203.15; 239/8

(57) ABSTRACT

A method of creating small particles by a technology referred to here as “violent focusing” is disclosed, along with devices for generating such violent focusing. In general, the method comprises the steps of forcing a first fluid out of an exit opening of the feeding channel to create a fluid stream. The exit opening is positioned such that the fluid flowing out of the channel flows toward and out of an exit orifice of a pressure chamber which surrounds the exit opening of the feeding channel, and is filled with an atomizing fluid. An atomizing fluid such as a gas is directed towards the first fluid stream in approximately orthogonal directions and surrounding the circumference of the first fluid stream from all sides. The first fluid flow is broken into particles which have dimensions which are smaller than the dimensions of this fluid stream.

### CROSS-REFERENCES

This application is a continuation-in-part of application Ser. No. 09/591,365 filed Jun. 9, 2000 which claims priority to earlier filed provisional application Ser. No. 60/138,698 filed Jun. 11, 1999, which applications are incorporated herein by reference in their entirety.

### FIELD OF THE INVENTION

This application generally relates to the creation particles created by the directed flow of fluids.

### BACKGROUND OF THE INVENTION

Devices for creating finely directed streams of fluids and/or creating aerosolized particles of a desired size are used in a wide range of different applications, such as, for example, finely directed streams of ink for ink jet printers, or directed streams of solutions containing biological molecules for the preparation of microarrays. The production of finely dispersed aerosols is also important for (1) aerosolized delivery of drugs to obtain deep even flow of the aerosolized particles into the lungs of patients; (2) aerosolizing fuel for delivery in internal combustion engines to obtain rapid, even dispersion of any type of fuel in the combustion chamber; or (3) the formation of uniform sized particles which themselves have a wide range of uses including (a) making chocolate, which requires fine particles of a given size to obtain the desired texture or “mouth feel” in the resulting product, (b) making pharmaceutical products for timed release of drugs or to mask flavors and (c) making small inert particles which are used as standards in tests or as a substrate onto which compounds to be tested, reacted or assayed are coated. There are numerous ways of finely breaking up an fluid (typically, a liquid, an emulsion, or a suspension or a slurry of particles suspended in a liquid) into droplets. Referring to this fluid as the first fluid, the present invention pertains to a class of methods in which a second fluid provides the energy necessary to finely divide and disperse the first fluid into smaller fragments or particles. Two characteristics of the size distribution of the particles are generally sought: an average particle size, and a dispersion or variability of particle sizes, both of which are tuned to meet the requirements of a particular application. In addition, the energy consumption per unit mass of the first fluid, and the proportion of first and second fluid masses are also of paramount importance, as are the durability, manufacturability, and cost of a particular atomizer design.

In a carburetor of a piston engine with spark ignition, the liquid is atomized finely to enhance evaporation of the fuel, and subsequent combustion (Bayvel-Orzechowski, 1993, page 199). In pulmonary drug delivery via an aerosol, particles with a mass median aerodynamic diameter typically between 0.5 and 6 micron are required. In this application, the goal is to generate small enough particles so that they can be transported to the lung of the patient via inhalation, and deposited in the desired region of the lung by inertial impaction or gravitational sedimentation, with smaller particles depositing more peripherally.

Methods in which this second fluid is a gas and the first fluid is a liquid have a long history. They are known as “pneumatic atomization”, also as “two-fluid atomization” (Gretzinger-Marshall, 1961), and as “twin fluid” atomization. Pneumatic atomization has been reviewed by Lefebvre (1989), and by Bayvel-Orzechowski (1993). The first fluid to be atomized (a liquid) is generally passed through a passage or channel and out of an exit into a region in which the liquid encounters and interacts with the atomizing fluid, a gas. The exit end of the channel is thus positioned such that the liquid coming out of such end encounters gas moving at sufficient velocity to allow atomization to take place. Pneumatic atomizers are widely used in applications in which a source of compressed gas exists, and good dispersion of the particles within the gas is desired. Some examples are molten metal atomization for the production of metal strip (Layernia-Wu, page 21), and fuel oil atomization in boiler furnaces. In the first example, the goal is to obtain the right metal droplet size at reduced cost, but the droplets must typically be heavy enough to deposit, gravitationally or by inertial impaction for example, on a substrate. In the second example, the object is to generate as small a particle as possible so that it can evaporate or have enough surface area for the combustion to proceed to as nearly to completion as possible, to avoid wasting fuel, and releasing incompletely oxidized fuel into the environment.

Pneumatic atomizers have been classified according to low-, intermediate-, or high-gas pressure (Table 4-3 in Bayvel-Orzechowski, 1993, p 196). They have also been classified considering the direction of gas action on the liquid (Bayvel-Orzechowski, 1993 page 197.) In “swirl-flow atomizers”, one of the two fluids is subjected to swirling before it encounters the other fluid. In “parallel-flow atomizers”, the liquid flow is in the same mean direction as the gas at the moment of encounter. Examples of this type are so-called “concentric nebulizers” and “convergent atomizers” (such as in pat. U.S. Pat. No. 6,166,379, widely used for inductively coupled plasma mass spectrometry, ICP-MS; or as shown in Gretzinger-Marshall, 1961.) In “cross-flow atomizers”, liquid jets are introduced into a gas stream, commonly at 90 degrees from a single direction, although angles smaller and greater than this have been used (Bayvel-Orzechowski, 1993 pages 199-204.) Cross flow atomizers with external action (i.e. where the gas is impinged on a liquid jet outside the nozzle) are widely used for the atomization of molten metal (Layernia-Wu, 1996). The active participation of the air during the disintegration process distinguishes pneumatic methods from (non-pneumatic) methods in which the gas flow only serves to disperse the droplets resulting from the spontaneous disintegration of liquid jets by capillary instability, thus preventing droplet coagulation or impaction on solid walls (Schuster et al. 1997). However, the air can participate to varying degrees.

Pneumatic atomizers are also referred to by the terms “Air-assist” and “Airblast”. The distinction made is that in air assist atomization, there is a source of high pressure gas, while the air velocity in an airblast atomizer is usually limited to 120 m/s. Thus, air assist atomizers are characterized by a relatively small quantity of high velocity air, while airblast atomizers use a higher quantity of limited velocity air. Airblast atomizers are used in aircraft, marine and industrial gas turbines. The need for an external supply of high pressure air, for example, has ruled out air-assist atomizers for aircraft applications. (Lefebvre, 1989, chapter 4)

Gas can participate in creating atomization in a mechanistically different way from traditional pneumatic methods. This is what occurs in the so called “flow focusing” method, in which a fluid flows out of a chamber through an orifice, and a tube inside the chamber and supplying a slow stream of another fluid, which is immiscible in the first fluid, is brought towards the orifice through which a first fluid is exiting the chamber. As the first fluid exits the end of the tube, it senses the pressure gradients that have set up in the flow of the other fluid, and gets accelerated towards the center of the orifice under the influence of those pressure gradients, thus attaining a very small stream width. The break up of the resulting thin stream of first fluid can proceed via normal Rayleigh capillary instability. [U.S. Pat. No. 6,119,953 and other U.S. patents to Ganan-Calvo.

### SUMMARY OF THE INVENTION

A method of creating small particles, aerosols, and hydrosols, by a technology referred to here as “violent focusing” of a fluid, to break up and disperse said fluid is disclosed, along with devices for generating such violent focusing. The fluid to be atomized (first fluid) exits from a supply means. A second fluid, a gas for the generation of aerosols, or a liquid for the generation of hydrosols, emulsions, and micro-bubbles, surrounds the exit of the supply means, and is directed with a high speed onto the first fluid in the region immediately outside and in front of said exit. Immediately before encountering the first fluid, the direction of flow of the second fluid is substantially orthogonal to the stream of first fluid, and the width of the stream of second fluid directed towards the first fluid is similar or smaller than the width of the first fluid stream at the exit of the first fluid supply means. The action of the second fluid on the first fluid is to cause a focused stream of first fluid to breakup into small particles, arising both from the pressure gradient forces and shear stresses that the second fluid exerts on the first fluid. During the process of atomization, the speed of the stream of second fluid is higher than the speed of the first fluid stream. In general, the technique can be expanded to three, four, or any number of fluids. For example, the second fluid can be used to form a concentric cylinder around the stream of the first fluid which stream disassociates resulting in encapsulation of the particles of the first fluid, and the third fluid can be a gas for aerosolizing the encapsulated particles, or a liquid for providing a hydrosol of the encapsulated particles. Such techniques would have utility in the generation of for example, timed release formulations of pharmaceuticals for injection or inhalation. Examples of appropriate encapsulation media include, but are not limited to liposomes, polymers, or glycols.

While pneumatic methods have inherent advantages, successful applications of pneumatic atomization depend on proper management of the inherent disadvantages of this form of atomization. Pneumatic atomizers are disadvantageous relative to non-pneumatic forms of atomization in their need of a source of compressed gas, as well as in their generally higher requirements of energy used to atomize a unit mass of liquid. This higher energy usage is recognized to be associated with the need to compress gas, but is also associated with a general low efficiency of energy transfer. Another disadvantage associated to pneumatic atomizers is their relatively complex geometry/structure, which makes them more expensive to manufacture. (Bayvel-Orzechowski, 1993, page 195)

The needs for improving energy efficiency and for reducing design complexity are usually conflicting. For example, in order to improve energy exchange between the gas and the liquid, atomizers with a complicated design that allows combined internal and external exposure of the liquid to the air have been devised (FIG. 4-48, and Ref 24 in Bayvel-Orzechowski, 1993, p 199). In another example, Jennings in U.S. Pat. No. 3,463,404 teaches a system for maintaining good atomization at a variety of liquid flows. While this system is simple in design, it requires incurring large energy losses associated with forcing the gas through a porous plug in the region immediately preceding the region of encounter of the gas with the liquid.

Energy transfer is sometimes facilitated by providing a narrow passage for the air at the location where the two fluids meet. This has the effect of raising the local speed (and thus momentum) at which the second fluid encounters the first fluid, for a given total mass flow rate of second fluid available. Momentum is the driving force for these forms of atomization, with higher momentum leading to greater shear forces that breaks up the first fluid.

The air-liquid combination is just one of the fluid combinations that this disclosure is concerned with. Energy efficiency is managed in the present invention by a) avoiding excessive energy losses in the transfer of the fluids from their high pressure points in the supply lines to their point of encounter, and b) enhancing the efficiency of transfer of the energy from the atomizing fluid to the atomized fluid. These aspects are managed through proper configuration of a simple atomizer geometry. According to the invention, the energy and momentum transfer from the air to the liquid is improved, so that the desired particle size distribution can be achieved with a smaller consumption of energy. Alternatively, for a given consumption of energy, the particle size is reduced. This improved transfer of energy and momentum is achieved by properly arranging the surfaces confining the liquid and the gas.

The invention disclosed has the added advantage of ease of manufacture. In addition, the simplicity of the geometry allows very small dimensions, thus allowing further reductions in the particle size by creating an atomizer with reduced dimensions, which exposes a greater interfacial area of the first fluid to the second fluid per unit volume of first fluid. Thus, a distinct advantage of the invention is the simplicity of its geometry, which allows it to be produced in miniature size (e.g. less than one kilogram) inexpensively, as might be required for example, for pulmonary drug delivery applications. Another advantage is the ability to form aerosols of 1-3 micrometers in diameter, as required for efficient delivery of pharmaceuticals to the lungs. Miniature size atomizers can be easily stacked up or combined into a single unit to obtain a desired amount of delivered atomizate in a predetermined amount of time. This is particularly important when the overall size of the unit needs to be small, such as in pulmonary applications in which the object is to obtain a portable device having a small overall size. Another advantage of the geometry disclosed is in its very low deposition of particles on the solid walls of the atomizer.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic cross-sectional plan view of a nozzle of the two fluid embodiment of the invention, showing schematically the first fluid undergoing violent focusing atomization.

FIG. 2 is a close-up, cross-sectional view of the region of encounter of the first and second fluids in a generic embodiment, showing and labeling various angles, points, and areas of the nozzle (P. R. P′ refer to points of geometrically well defined position; angles are provided or labeled with Greek symbols);

FIG. 3 is another embodiment of the nozzle of FIG. 1 with various angles and areas labeled;

FIG. 4 is a similar embodiment of the nozzle of FIG. 1 with certain areas and angles labeled;

FIG. 5 is an embodiment of the nozzle of FIG. 1 with various parameters labeled;

FIG. 6 is a graph of the volume median diameter (VMD) against the first fluid supply flow rate for four different first fluids;

FIG. 7 is a graph of the dimensionless volume median diameter (VMD) versus dimensionless first fluid flow rate with a line through the data points showing the best power-fit;

FIG. 8 is a graph of the data with the line shown in FIG. 7 compared to a theoretical line for the Rayleigh breakup prediction of a flow-focused jet; and

FIG. 9 is a graph of the geometric standard deviation (GSD) against dimensionless first fluid flow rates obtained with the different liquids listed.

FIG. 10 is a graph of the 85% lower percentile diameter of the particle volume distribution against the channel width

FIG. 11 is a graph of the geometric standard deviation against the channel width

FIG. 12 is a graph of the same data shown in FIG. 10, plotted against the (dimensionless) ratio of channel width H over the first fluid supply means channel width Dₒ

### DETAILED DESCRIPTION OF PREFERRED EMBODIMENTS

Before the present aerosol device and method are described, it is to be understood that this invention is not limited to the particular components and steps described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”. “and,” and “the” include plural referents unless the context clearly dictates otherwise. Thus, for example, reference to “a particle” includes a plurality of particles and reference to “a fluid” includes reference to a mixture of fluids, and equivalents thereof known to those skilled in the art, and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference in their entirety to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “atomization” is used herein to mean any process by which a fluid is broken up into separate fragments or particles, typically from a fluid stream, which fragments or particles typically are much smaller than any dimensions of the stream or drop of fluid from which they detached.

The terms “atomizer” and “nozzle” are herein used to refer to one unit that is capable of atomizing a fluid using another fluid.

The terms “energy”, “pressure energy” and the like are used herein to mean mechanical energy in the form of kinetic energy, or of enthalpy of the fluids, and does not necessarily, but may include interfacial energy. The term “energy” is herein used sometimes to refer to the total energy used for pumping the fluids through the atomization nozzle during operation. The precise meaning of these terms should be clear from the context to anyone skilled in the art. The term “energy loss”, “frictional losses” and the like is used herein to mean the amount of mechanical energy that is transformed into internal energy through known energy dissipative mechanisms such as viscous action, gas compression through shock waves, etc. The terms “consumption of energy”, “energy consumption”, “energy use” and the like are used herein to mean the amount of energy used per unit time required to atomize a unit mass of first fluid (Btu/hr/lb), and generally amounts to the mechanical energy in the final mixture plus energy losses.

The terms “first fluid”, “first liquid” and the like are used herein to mean a fluid that is delivered out of a first fluid supply means into a region where it gets atomized, and in general is (although is not limited to) a single or multiple phase liquid. For example, single component liquid; or a multiple component liquid mixture (comprising one or more liquids and/or solutes); or a multi-phase liquid, such as an emulsion comprising one or more liquids emulsified into another liquid; or a suspension or slurry of solid particles or biological molecules, cells, or liposomes, suspended in a liquid matrix; or a supercritical fluid; or combinations of these fluid systems thereof. For pharmaceutical drug delivery, the first fluid will in general comprise an active drug or mixture of multiple active drugs, and pharmaceutically acceptable excipients.

The terms “particles”, “aerosol particles” and the like are used herein to mean the fragments of fluid or fluids atomized. The term “particle suspension”, “atomizate” and the like are used herein to mean the collection of the fragments of first fluid, usually after exiting the nozzle (also called “atomizer”), and in suspension in a matrix of atomizing fluid.

The terms “second fluid”, “second liquid” and the like are used herein to mean the fluid directed at the first fluid to accomplish atomization, and in some embodiment of the invention, to accomplish such processes as encapsulation of the first fluid. The second fluid can be (but is not limited to) a liquid, or a gas, emulsion, suspension, or a supercritical fluid. It will be obvious to one skilled in the art the second fluid can contain many components, such as the components listed for the first fluid, or such things as sugars, polymers or lipids for encapsulation, glycols including but not limited to poly(ethylene-glycol), or any number of other compounds. Preferred compounds for encapsulation include, but are not limited to poloxymers, including polyoxyethylene, gelatin, and in the preferred embodiment for pharmaceutical encapsulation is poly(lactic-co-glycolic) acid

The term “pressure chamber” is used herein to describe a region of the nozzle, which receives atomizing fluid at high pressure through a supply means and channels this fluid through a channel into substantially all areas surrounding the first fluid immediately exiting a first fluid supply means, and discharges first and second fluids through a discharge orifice.

The terms “exit orifice”, “discharge orifice”, “discharge opening” and the like are used herein to mean the passage through which the first and second fluids are discharged out of the pressure chamber.

The terms “first fluid supply means”, “feeding supply means”, and the like are used herein to describe a structure that has passages for supplying first fluid from a reservoir to a specified location in the pressure chamber, which means is typically in the form of a tube, although in general can have any shape, including but not limited to non circular cross sections, ovals, rectangles, concial ends or narrowing funnel shaped tapers

The terms “violent mode”, “violent focusing”, “violent atomization”. “violent focusing atomization” and the like refer to the process of atomization of a first fluid by the action of a second fluid which involves impinging of the second fluid onto the first fluid in all directions substantially orthogonal to the mean motion of first fluid, that results in both a narrowing of the first fluid stream, and in a breaking up of the stream into particles and the particles into smaller particles of first fluid. It may also involve a vena contracta of the second fluid.

General Methods

The method is carried out by forcing a first fluid through a first fluid supply means, e.g., a tube. The fluid exits the supply means into a pressure chamber filled with a second fluid. The chamber has an exit port preferably positioned directly in front of and downstream of the flow of first fluid exiting the first fluid supply means. A channel inside the pressure chamber directs the second fluid into trajectories that converge towards the exit of the first fluid supply means from all sides e.g. all around the circumference of exit of the first fluid supply means. Downstream from the channel, the two fluids interact and exchange energy, which exchange results in the narrowing and atomization of the first fluid. This turbulent interaction of the two fluids is generally referred to here as “violent focusing.” The first place of encounter of the two fluids is inside the pressure chamber, immediately in front of the exit of first supply means, and directly upstream from the exit of the pressure chamber. The direction of motion of the second fluid when it encounters the first fluid is approximately orthogonal to the direction of the flowing stream of the first fluid at the exit of its supply means. For example, when the first fluid supply means has symmetric cylindrical geometry, the second fluid in the second fluid channel radially converges toward the axis of cylindrical symmetry of the first fluid supply means. The channel of the second fluid may narrow in the direction of first fluid motion, and is preferably unobstructed by other solid or porous surfaces connecting both walls of the channel.

The “violent focusing” method comprises the steps of:

a) forcing a first fluid through a feeding supply means and out of an exit opening of said feeding supply means as a fluid stream.

b) continually filling a pressure chamber with a second fluid surrounding the exit opening and fluid stream fed therefrom.

c) forcing the second fluid through a channel inside the pressure chamber and out of the channel, in such a way that the second fluid stream exiting the channel is directed at the first fluid stream circumference in all directions of flow which are substantially orthogonal to the mean direction of flow of the first fluid stream exiting the first fluid supply means; i.e. the second fluid flows towards the first fluid from all sides at substantially orthogonal angles.

d) allowing the first fluid to be focused under the pressure and shear forces exerted by the convergent flow of the second fluid, outside and directly in front of the first fluid supply means; while allowing the second fluid stream to flow faster than the first fluid stream,

e) allowing the second fluid to break up the first fluid stream into particles that are substantially smaller than the width of the first supply means exit, and

f) allowing the first and second fluids to exit the pressure chamber through an exit port of the pressure chamber, positioned directly in front of the exit of the first fluid supply means.

The exit opening of the first fluid supply means preferably has a diameter in the range of about 5 to about 10,000 micro-meters, more preferably about 15 to 300 micro-meters. The exit opening of the pressure chamber preferably has a diameter in the range of about 5 to about 10,000 micro-meters, more preferably about 15 to 400 micro-meters, and the exit opening of the supply means is positioned at a distance from the pressure chamber exit opening in a range of from about 5 to about 10,000 micro-meters, more preferably about 15 to about 300 micro-meters. In general, the width of the second fluid stream at the channel exit is less than 2 times the width of the first fluid supply means exit, preferably less than 1.5 times the width of the first fluid supply means exit, more preferably less than 1 times the first fluid supply means exit, and most preferably from 0.2 to 0.7 times the width of the first fluid supply means exit.

The first fluid can be (but is not limited to) a liquid, an emulsion, or a suspension or slurry comprising solid particles suspended in and/or partially dissolved in a liquid. The second fluid can be but is not limited to a liquid, a gas, or a supercritical fluid. The second fluid can in general be of any composition, including but not limited to liquids, suspensions, solutions, aerosols, supercritical fluids, but in the preferred embodiment is a gas or a fluid substantially immiscible in the first fluid. Any gas or gas mixture could be used, including but not limited to air, nitrogen, carbon dioxide, helium, argon, or any other acceptable gas or mixture of gasses.

Depending on the application, the two fluids may be immiscible or completely miscible, or miscible to varying degrees. For example, this invention can be used to enhance transport processes that are aided by an increased interfacial area between the two phases, including dissolution of poorly miscible liquids, or evaporation of a liquid first fluid (e.g. fuel) into a gaseous second fluid (e.g. air). For encapsulation applications, in general the two fluids will be immiscible or poorly miscible. There may be applications where one or both of the fluids are mixtures of components, some of which are miscible and some of which are immiscible in one or several of the components of the other. It will be obvious to one skilled in the art that many combinations of miscibility/immiscibility could have utility.

The walls that define the channel leading the second fluid or gas to the stream of the first fluid do not have to be connected. However, these walls generally present a clear path for flow of the second fluid to the stream of first fluid. While said walls may be connected inside the channel by solid objects such as (but not limited to) porous objects, ribs, fins, etc., the channel preferably comprises an open passage. Having an open channel minimizes energy losses as the second fluid flows through the channel, allowing for a more efficient process. The pressure driving the flow of second fluid is such that sufficiently high velocity is imparted on the second fluid at the exit of the channel to bring about the atomization. The flow of second fluid encounters the first fluid in the pressure chamber at an angle to the direction of flow of the first fluid inside the supply means near the exit which is preferably equal to about 90 degrees +/−about 45 degrees, preferably 90 degrees +/−about 30 degrees, still more preferably 90 degrees +/−about 15 degrees, most preferably 90 degrees +/−, 5 degrees.

The separation between the walls defining the channel determines the amount of mass of second fluid consumed given a velocity of second fluid, and thus affects the quantity of energy spent. However, the present invention is a particularly advantageous configuration in terms of energy use, and therefore allows a separation between the walls that is quite small, and in general comparable to the width of the first fluid supply means.

Based on the above it will be seen that very small particles can be created, and that particles much smaller than the dimensions of the first fluid supply means and the channel can be created. This can be very important for many applications. For example, for the pulmonary delivery of drugs, particles in the range of 0.1 to 10 micro-meters are required, and for efficient delivery particles of 0.5 to 6 micro-meters, or preferably 1 to 3.5 micro-meters, are required. When the dimensions of the supply means are comparable to the dimensions of the particles, blockage of the very small structures can occur. This problem can be reduced or obviated by the current invention. In addition, the relative large dimensions of the supply means and channel will allow for efficient delivery of suspensions.

The invention can in general be expanded to include a third, fourth, fifth, or any number of fluids, each similar to the previously described first fluid or second fluid, wherein if it is similar to the previously described first fluid, its supply means will in general be concentrically positioned around and containing the first fluid supply means and the flow will be parallel to the first fluid. Thus, a cylinder in a cylinder, etc. If it is similar to the previously described second fluid, it will comprise a distinct channel for directing said fluid toward the exit of the previous fluid's pressure chamber. These subsequent fluids can have any of the properties of the first and second fluids disclosed above. For example, the first fluid could comprise a formulation containing a pharmaceutically active compound, the second fluid could be used to coat or encapsulate particles of said formulation, and the third fluid could be a gas used to disperse said coated or encapsulated particles as an aerosol. Any number of fluids could be used to create any number of desirable properties. It is also possible to use a first and second fluids in the nozzle which then discharge out of the nozzle into a bath of a third fluid.

To help better appreciate the importance of atomizer design in the atomization of fluids, we note that the energy spent in accelerating the second fluid to the site of atomization increases directly with the total momentum carried by that fluid at that location. It is reminded that atomization is achieved when such momentum is transferred effectively to the first fluid, resulting in its breakup. Therefore, the key to not wasting energy unnecessarily lies in making use of the available momentum (that carried by the second fluid) for atomizing first fluid to the extent possible, and that is done through adequate atomizer design. In other words, increasing the extent of atomization can be done by the brute force method of increasing the total momentum in the second fluid. However, this approach results in a proportional increase in the energy spent.

To support the claim that the energy is proportional to the total momentum, we present the following simplified analysis, which considers incompressible fluids. A pump or pressure source of second fluid provides the energy consumed for the process (the first fluid generally carries a negligible source of energy). The energy consumed by the pump or the pressure source is equal to the work per unit time, K, which is the product of the pressure p times the flow rate of second fluid Q:
K=pQ

p is generally measured relative to a point in the system, taken here to be the site of atomization. In the absence of viscous losses, the Bernouilli theorem allows us to express p in terms of the velocity at the site of atomization, p is in fact equal to 0.5 times the momentum flux at the site of atomization (Kg/m/s²):
p=0.5×(momentum flux)=0.5(ρV²)

Here ρ is the density of the second fluid and V its velocity (assumed uniform at the site of atomization). Q is the volume rate associated with the flow of second fluid. Because an incompressible second fluid is considered, its density is constant, and the volume rate at the pump is the same as the volume rate at the site of atomization, expressed as the product of the cross sectional area A which the second fluid flows through with velocity V at the site of atomization:
Q=VA

Combining the expressions for Q and for p, the energy spent can be expressed as
K=0.5(ρV²)VA

The total rate of momentum P carried by the second fluid at the site of atomization is expressed in units of (kg m/s²), and is represented by the product of the total mass per unit time Qρ (kg/s) of second fluid times its momentum per unit mass or speed V (m/s) at the site of atomization. Thus,
P=ρQV=ρV²A
and
K=0.5P^{3/2}/(ρA)^{1/2}

It can thus be seen that raising the momentum P by applying more pressure at the pump, will result in more units of momentum carried at the site of atomization. Although this will result in more units transferred to the first fluid, it will also result in an increase in the amount of energy spent K.

General Device

The basic device or nozzle of the invention can have a plurality of different embodiments. However, each configuration or embodiment will comprise a means for supplying a first fluid (preferably a liquid) and a means for supplying a second fluid (preferably a gas) in a pressure chamber which surrounds at least an exit of the means for supplying a first fluid. The first fluid supply means and pressure chamber are positioned such that mechanical interaction resulting in atomization of the first fluid takes place between the first fluid exiting the first fluid supply means and the second fluid exiting the supply chamber. The exit opening of the pressure chamber is downstream of and preferably it is directly aligned with the flow path of the means for supplying the first fluid.

To simplify the description of the invention, the means for supplying a first fluid is often referred to as a cylindrical tube. However, tube shape could be varied, e.g. oval, square, rectangular, and can be of uniform cross section or tapered. For example the exit of the first fluid supply means may be a slit defined by two walls or surfaces, and having a long dimension and a short dimension. The first fluid can be any fluid depending on the application. For example, the fluid could be a liquid formulation comprising a pharmaceutically active drug used to create dry particles or liquid particles for an aerosol for inhalation, suspensions for injection, or other pharmaceutical applications. Alternatively, it could be a hydrocarbon fuel used in connection with a fuel injector for use on, for example, an internal combustion engine, turbine, heater, or other device which burns hydrocarbon fuel. In general, the first fluid could be (but is not limited to) a single or multiple phase liquid. For example, it can be a single component liquid; or a multiple component liquid mixture (comprising one or more liquids and/or solutes); or a multi-phase liquid, such as an emulsion comprising one or more liquids emulsified into another liquid; or a suspension or slurry of solid particles or biological molecules, cells, or liposomes, suspended in a liquid matrix; or combinations of these liquid systems thereof. The second fluid can be any fluid, as described previously, but preferably is a gas and that gas is generally air or an inert gas, such as carbon dioxide, or gas mixtures of inert gases. The two fluids are generally immiscible or mildly miscible. However, on some applications, violent focusing can be used to enhance mixing between two poorly miscible fluids or phases, thanks to the large interfacial area between the two phases of fluids that is created during violent focusing.

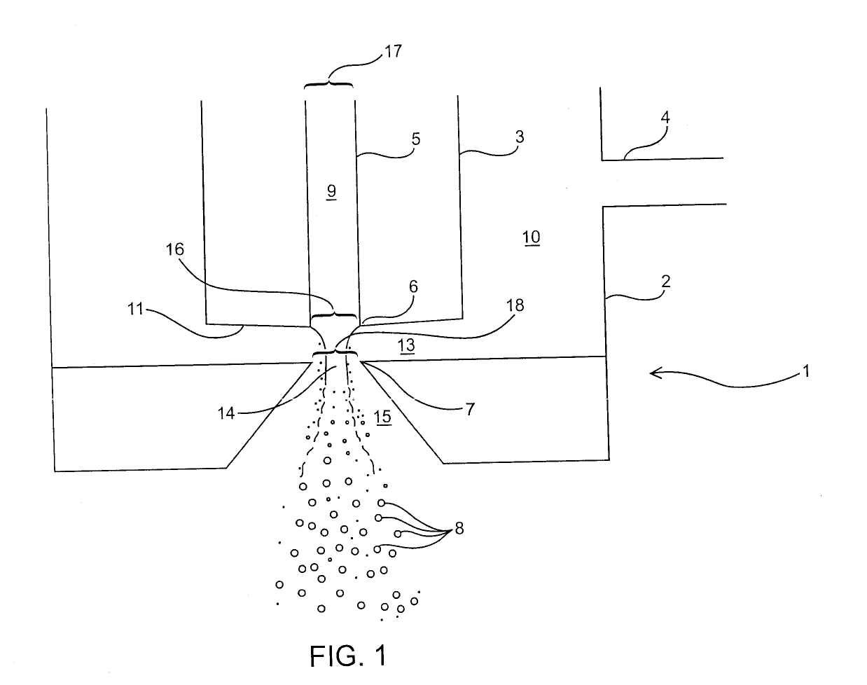

An example is dissolution of poorly miscible liquids. Another is evaporation of fuel into air or another oxidizing gas e.g. oxygen. Here evaporation can be viewed as a form of mixing of a liquid's constituent molecules into a gaseous solvent, the oxidizing atmosphere. It is possible to have situations wherein the liquid upon exiting either the first fluid supply means or the pressure chamber vaporizes to a gas on exit. Such is not the general situation. Notwithstanding these different combinations of liquid-gas, and liquid-liquid, the invention is generally described with a liquid formulation being expelled from the supply means and interacting with surrounding gas flowing out of an exit of the pressure chamber. Further, the exit of the pressure chamber is generally described as circular in cross-section and widening in a funnel shape (FIG. 1), but could be any configuration, such as cylindrical, or have other shapes consistent with an entrance and an exit, which entrance represents the exit point of the pressure chamber.

Referring to the figures a cross-sectional schematic view of the nozzle 1 is shown in FIG. 1. The nozzle 1 is comprised of two basic components which include the pressure chamber 2 and the first fluid supply means 3. The pressure chamber 2 is pressurized by the second fluid 10 flowing into the pressure chamber via the entrance port 4. The first fluid supply means 3 includes an inner wall 5 defining an inner passage wherein the first fluid 9 flows. The first fluid supply means 3 can have any composition and configuration, including layers of dissimilar materials, voids, and the like, but is preferably a tube constructed of a single material. The inner wall 5 of the fluid supply means 3 is preferably supplied with a continuous stream of a first fluid 9 which first fluid 9 can be any liquid or gas but is preferably in the form of a liquid, suspension, or emulsion.

The pressure chamber 2 is continuously supplied with a pressurized second fluid 10 which can be any liquid or gas but is preferably a gas, or a supercritical fluid. The inner wall 5 of the first fluid supply means 3 includes an exit point 6. The pressurized chamber 2 includes an exit point 7, which marks the entrance to the discharge opening 15. The exit point 7 of the pressure chamber is preferably positioned directly downstream of the flow of first fluid exiting the exit point 6. The pressure chamber 2 includes channel 13 surrounding the exit 6 of supply means 3. The first fluid supply means exit 16, the channel 17, and the exit 18 of the pressure chamber 2 are configured and positioned so as to obtain two effects (1) the dimensions of the stream exiting the first fluid 9 supply means 3 are reduced by the second fluid 10 exiting the channel so that a focused stream 14 is formed; and (2) the first fluid 9 exiting the first fluid supply means 3 and the second fluid 10 exiting the channel 13 undergo a violent interaction to form much smaller particles 8 than would form if the stream of first fluid in reduced dimensions underwent normal capillary instability, e.g. formed spherical particles approximately 1.89 times the diameter of the first fluid stream.

The position of the exit port 18 could be in any location that allows the efficient “violent mode” atomization of the first fluid and efficiently delivers the resulting particles, but preferably, the exit port 18 of the chamber 2 is substantially directly aligned with the flow of first fluid exiting the first fluid supply means 3. An important aspect of the invention is to obtain small particles 8 from the interaction of the first fluid 9 and the second fluid 10, the first fluid 9 flowing out of the exit port 16 of the first fluid supply means 3. The desired formation of particles 8 is obtained by correctly positioning and proportioning the various components of the first fluid supply means 3 and the pressure chamber 2 and thus correctly proportioning the channel 13 as well as the properties of the fluids, including but not limited to the pressure, viscosity, density and the like, determining the mass flow, momentum flow, and energy flow of the first fluid fluids which flows out of both the first fluid supply means 3, of the second fluid which flows through the channel 13, and of the resultant mixed flow of combined streams of first and second fluids that flow out of exit 18, the result being particles 8. Specifically, there are some important geometric parameters that define the nozzle 1 of the present invention. Those skilled in the art will adjust those parameters using the information provided here in order to obtain the most preferred results depending on a particular situation.

Preferably, the first fluid 9 is held within an inner wall 5 which is cylindrical in shape. However, the inner wall 5 holding the first fluid 9 may be tapered (e.g. funnel shaped) or have other varying cross section, asymmetric, oval, square, rectangular or in other configurations including a configuration which would present a substantially planar flow of first fluid 9 out of the exit port 16. Thus, the nozzle of the invention applies to all kinds of configurations that have a channel for the second fluid 10 surrounding the first fluid means exit 16. Accordingly, the figures, including FIG. 1, are used only to define the variables but are not intended to imply any restrictions on the type of geometry or the specific details of the design of the nozzle 1 of the present invention. There are many degrees of freedom of design. For example, corners which are shown as sharp could be rounded or finished in different ways. Similarly, solid surfaces which are shown straight in the figures, could be curved, and could be patterned or admit different types of finishes, in order to obtained certain additional effects or optimize the design.

The focusing of the stream of first fluid 9 and its ultimate particle formation are based on the violent focusing experienced by the first fluid 9 on passing through and out of exit 16 and through exit 18 of the pressure chamber 2 which holds the second fluid 10.

Without being limited to any one theory, creation of particle 8 may occur as follows. The particular arrangement of the channel 13 causes a focusing of the first fluid 9 stream, as well as possibly a vena contracta of the second fluid stream, and a breaking up of the fluid stream into particles:

A) Focusing of first fluid 9 stream: The second fluid 10 attains a large momentum per unit volume in channel 13 exit at points 6 and 7. This rate of momentum flow can be described by the total momentum carried by the second fluid 10 per unit time at the exit of channel 13, which can be expressed in units of (kg/s) times (m/s), and be estimated as the product of the total mass flow rate (kg/s) of second fluid times the average speed (m/s) of second fluid at channel exit (defined in FIG. 1 by points 6 and 7).

Because a momentum per unit time received by an object represents a force on that object, and the second fluid 10 stream is incident on the first fluid 9, a portion of said rate of momentum flow is experienced by first fluid 9 as a force exerted by the second fluid 10 exiting channel 13. However, the net momentum of the whole of the second fluid 10 stream exiting channel 13 has a vectorial sum of zero or nearly zero in the plane of second fluid motion, because said fluid flow toward the first fluid 9 is evenly distributed around all sides surrounding the first fluid 9. Each portion of second fluid 10 having a specific direction of motion in channel 13 carries momentum, and thus exerts a force on the portion of first fluid 9 that said portion of second fluid impinges on. The net effect is a distributed force onto each portion of first fluid 9 towards inwards, resulting in a squeeze inward of the first fluid 9 stream. Such squeezing actions combined with the steady supply of first fluid results in a focused stream 14 of first fluid 9, such as the one illustrated in FIG. 1. In addition, the second fluid creates shears on the first fluid as it rushes over that first fluid. Such shear forces also tend to accelerate the first fluid away from the first fluid supply means, and this acceleration thus also tends to reduce the cross section of the first fluid 9 stream, as shown by focused stream 14 in FIG. 1.

B) Vena contracta of second fluid: As the streamlines of second fluid 10 that graze point 7 and bound the second fluid stream leave channel 13, a component of their velocity points towards the first fluid 9, and these streamlines detach from the walls of the channel at exit point 7. After detachment, the stream of second fluid 10 slows down in the direction of the channel 13 and accelerates in the axial direction (first fluid 9 flow direction), but the total width of the second fluid 10 stream surrounding the first fluid 9 stream becomes narrower than the width of the pressure chamber exit port 7. We are referring to this reduced cross section as the vena contracta of second fluid. As a result of the reduced cross section associated with such vena contracta, the average speed of second fluid is greater than the lower speed that would result if the second fluid stream could fill the entire width of the pressure chamber exit port 7. This augmented speed is associated with an augmented flow of momentum, and therefore, is more effective than a lower speed at breaking up the first fluid 9 into particles 8.

Based on the above it will be understood that when a vena contracta of second fluid 10 is present, configuring the system to have an angle between the second and first fluid streams of about 90 degrees has the advantage over other configurations having a much smaller angle. It will be further understood that the stream of second fluid carrying a desired speed and momentum is significantly narrower in width than either the pressure chamber exit port or the first fluid exit port.

C) Breaking up of first fluid into particles: Except for a very thin viscous boundary layer of fluid adjacent to the interface of first fluid 9 exposed to second fluid 10, the second fluid 10 flowing over the first fluid 9 does so at a faster speed than the first fluid 9. This difference creates a shear force in the direction parallel to the interface. This shear force tends to create undulations or waves on the surface of the first fluid 9, which grow and ultimately break off, resulting in fragments that detach from the main body of first fluid 9. Such fragments can themselves undergo subsequent fragmentation upon further interaction with the second fluid 10, and other fragments or portions of first fluid 9. Even under steady conditions of the flows of first and second fluids in their respective supply means, it is nearly inevitable that these instabilities and unsteady flows will take place, including an instability in which said viscous boundary layer becomes turbulent. Actions A), B) and C) described above can take place concomitantly, partially concurrently, or separately.

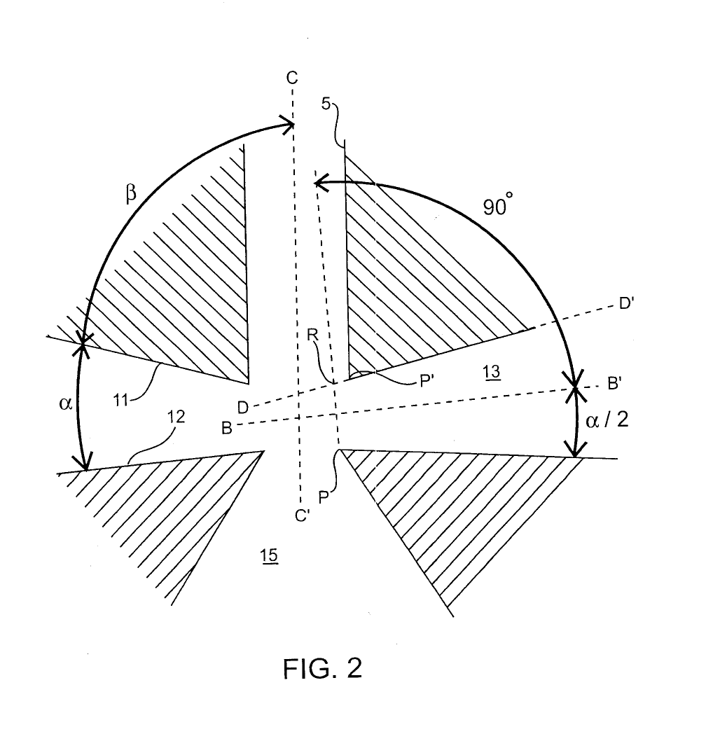

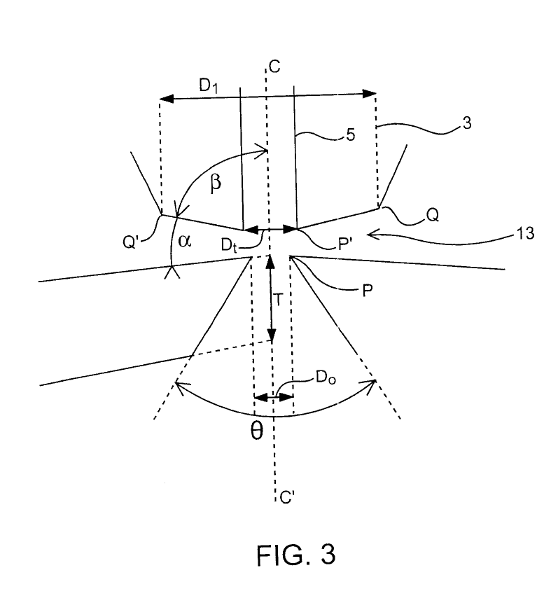

We refer now to FIGS. 2 and 3 in order to describe the relationships between some of the components shown in FIG. 1. First, a dashed line C--C′ is shown running through the center of the exit port 16 in which the first fluid 9 flows as well as the exit port 18 of the chamber 2. In symmetric planar atomizers, for example, the line C--C′ represents the plane of symmetry intersected by the plane of view. In cylindrically symmetric atomizers, this line represents the axis of cylindrical symmetry. The dashed line B--B′ represents the bisector of the second fluid channel 13 near its exit end. The area that has been referred to as the second fluid “channel” 13 is the open passage that lies in between the terminal face 111 of the first fluid supply means 3 and the front face 12 of the chamber 2. The exit of the channel 13 is defined by edges P and P′ (appearing as points on the cross sectional view of FIGS. 2 and 3). However, the width of the second fluid stream upon exiting channel 13, also called “channel exit width”, or simply “channel width”, is taken as the distance between points P and R of FIG. 2, and is also referred to by symbol H. Other geometric parameters of critical importance are Dₜ, which is the first fluid exit width; Dₒ, which is the width of the pressure chamber exit; and the channel length, which will be quantified using the related parameter Dₗ, which is the full width of the first fluid supply means defined as the full separation between channel entrance points Q and Q′ as shown in FIG. 3. In order for violent atomization to take place, these angles and certain ratios of these dimensions must be satisfied, as will be described and quantified in the following.

To obtain desired results with the nozzle of the present invention the following characteristics must be present:

(a) a strong convergence of the streamlines of the second fluid 10 (liquid or gas) in the chamber 2 towards and surrounding the first fluid 9 coming out of the exit port 16;

(b) efficient utilization of the momentum in second fluid 10;

(c) a focusing or narrowing of the stream of the first fluid 9 by the surrounding fluid 10 from the channel 13.

The above characteristics (a)-(c) combine with each and with other characteristics in order to result in the desired (d) violent focusing of the stream of fluid 9 exiting the exit port 16. For example, other characteristics may include the fluid 9 and/or 10 obtaining sonic speeds and shock waves (e) when the second fluid 10 is a gas, and may also include a vena contracta of the second fluid stream after it has come in contact with the first fluid stream.

In order to more fully understand the invention, each of the characteristics (a)-(e) referred to above are described in further detail below.

(a) Strong convergence of second fluid:

The primary characteristic of the present invention is the facilitation of a strongly convergent (imploding) flow of second fluid 10 towards and surrounding the first fluid 9. The fluid 10 in the pressure chamber 2 should preferably not flow parallel to the first fluid 9 exiting the first fluid supply means, i.e. the two fluids should preferably not intersect at a 0 degree or small angle. Further, the second fluid 10 in the pressure chamber should preferably flow substantially directly perpendicular to, or with a similar large angle relative to the flow of, the first fluid stream 9 exiting the first fluid supply means 3.

In order to generate significant convergence in the second fluid 10 toward the first fluid 9, the second fluid 10 should be admitted into a path that directs it towards the first fluid at a high angle. Specifically, the following design constraints based on the parameters shown in FIGS. 2 and 3 are preferably:

(1) a second fluid channel tapering angle α smaller than 90 degrees, preferably smaller than 30, more preferably between 0 and 10 degrees, but a is most preferably about 0 degrees.

(2) the wall 11 of the channel 12 should form an angle β (FIGS. 2 and 3) with center line C--C′ greater than 45 degrees but smaller than 135 degrees, preferably between 75 and 105 degrees, and most preferably, of about 90 degrees; and

(3) the length of the second fluid channel 13, defined as the distance between points Q and P′ (shown in FIG. 3), should be adjusted based on the other factors. The channel 13 should be long enough to facilitate the bending of the streamlines of second fluid 10 towards a path defined by the channel bisector B--B′, which is substantially orthogonal to the first fluid 9 flow direction. Thus, in general, Dₗ is required to be at least equal to 1.5 times the greater of Dₒ and Dₜ, and is preferably more than 1.5 times the greater of Dₒ and Dₜ, most preferably more than 2 times the greater of Dₒ and Dₜ. However, the channel 13 should not be so long that frictional losses between the second fluid and the walls of the channel become unacceptably high for the application in question, or so long that the viscous boundary layer becomes turbulent in the channel. This requirement also depends on other properties, generally combined into a Reynolds number. Those skilled in the art, reading this disclosure will be able to determine which combinations of those parameters lead to unacceptably high losses in a particular application.

(b) Efficient Utilization of Second Fluid Momentum:

To ensure efficient utilization for atomization of the momentum that the second fluid 10 carries at the point where it meets the first fluid 9, two independent conditions should be satisfied for two ratios involving geometric parameters defined earlier, namely Dₜ/Dₒ and H/Dₒ. H is a measure of the width of the exit of the channel 13, and equals the distance between points R and P in FIG. 2. Dₒ is the width of the pressure chamber exit, and Dₜ is the width of the first fluid supply means exit. In general, none of these three dimensions can be much greater or smaller than the other two. For example, a very large Dₒ in comparison to Dₜ (regardless of H) would, for example, permit the escape of second fluid and the corresponding momentum from the pressure chamber through regions of its exit port cross section that are far from the first fluid stream, and, therefore, the majority of the momentum carried by the second fluid 10 at the exit of the channel at point P would not be delivered towards, and utilized for shearing and atomizing, the first fluid 9. This underutilization of the momentum ultimately represents an unnecessary energy loss, which is avoided by the violent focusing method. On the other hand, it is conceivable for violent focusing to be able to take place for a Dₜ that is quite large compared to Dₒ, so long as H stays comparable to Dₒ. The ratio of Dₜ/Dₒ should be greater than 0.5 and preferably between 0.7 and 1.2, and most preferably between 0.8 and 1.0. It is worth noting that values under unity allow for visual inspection of the alignment of the first fluid channel from a line of sight from outside the nozzle into the pressure chamber exit port, and thus present a manufacturing advantage over ratios greater than unity.

The efficient utilization of momentum of the second fluid 10 also depends on the ratio H/Dₒ. This ratio governs where in the second fluid flow path (which includes the channel exit of width H, and the pressure chamber exit of width Dₒ) the speed of second fluid reaches its highest value. In general, the narrowest cross section in said flow path carries second fluid at, or approximately near, the highest speed. For example, when H/Dₒ is close to unity, both the exit of the channel and the pressure chamber exit carry second fluid at or near the maximum speed attained along said flow path. However, if H/Dₒ had a value much greater than unity, then the speed at the exit of the channel would be much smaller than the speed attained near the pressure chamber exit. This condition is undesirable, because the energy used to pump second fluid through the pressure chamber is employed to accelerate the second fluid inside the pressure chamber exit, thus after, rather than right before, it encounters the first fluid. Reducing width H would automatically cause an increase in speed, thus momentum, of the second fluid at the channel exit, right before it encounters the first fluid. This would be done with little change in the overall energy use, but with a great difference in the extent of atomization of the first fluid 9.

If, on the other hand, H/Dₒ had a value much smaller than unity, then the speed at the exit of the pressure chamber would be much smaller than at the channel exit. This condition is generally undesirable because maintaining the second fluid at high speed improves atomization during the exiting of the streams from the pressure chamber and ensures a thorough degree of atomization without an undue expense of energy. A very small H, could impact energy use also by bringing about unnecessary frictional losses in the channel, due to excessive friction between the second fluid 10 and channel walls 11 and 12. It should be noted however, that those losses are a function of other quantities, such as channel length, already discussed, or such as density (kg/m³) and dynamic viscosity (kg/m/s) of the second fluid. Systematic studies described under Examples for experiment 5 demonstrate that there is an optimum range of ratios of channel widths to pressure chamber exit width, for the particular set of conditions studied. In general, the width of the second fluid stream at the channel exit (H) is less than 2 times the diameter of the pressure chamber exit (Dₒ), preferably less than 1.5 times the width of the pressure chamber exit, more preferably less than 1 times the pressure chamber exit, and most preferably from 0.2 to 0.7 times the width of the pressure chamber exit.

In general, aside from the requirements on these geometric ratios, it is desirable to have as high a momentum as needed in the second fluid 10 for a certain amount of second fluid mass flow and for given conditions of pressure and temperature. The ratio between momentum and mass fluxes is similar to its average speed (in fact, is very nearly such value when variations in local speed are negligibly small across the second fluid channel exit). Both for compressible as well as incompressible fluids, the fastest speed is generally obtained in the narrowest part of the second fluid flow path, which includes the channel, the pressure chamber discharge opening, and the space in between where the two fluids first encounter each other. Again, if the distance between points R and P (FIG. 2) is too large, then the narrowest point in the flow path will be at the exit orifice. In the presence of the first fluid 9, such analysis implicitly assumes that the interface between not yet atomized first fluid 9 and second fluid 10, acts as part of the boundary that limits and defines the flow path for the second fluid. Thus, ignoring the space occupied by first fluid 9, a typical value of H compatible with the requirement of high speed at either the exit of the channel 13 or of the pressure chamber 2, is:
H=βDₒ

For axi-symmetric configurations, β equals 0.25; while for planar-two dimensional configurations, β equals 0.5. These values are consistent with the most preferred ranges for H/Dₒ provided earlier.

H must be large enough to preclude excessive friction between the second fluid 10 and the second fluid channel 13 walls that can slow down the flow and waste pressure energy (stagnation enthalpy) into heat (internal energy). An approximate guiding principle is that H should be greater than Hₘᵢₙ, defined as a few times the thickness of the viscous boundary layer δ_{L} that develops inside the second fluid 10 in its acceleration through the second fluid channel 13:
Hₘᵢₙ˜λδ_{L}
λ˜1 to 10

The thickness of the boundary layer at point P′ (FIG. 2) for the case when the second fluid is a gas and its speed is near the speed of sound (at the exit of the channel or at the exit of the pressure chamber) is approximately given by the following expression:
δ_{L}=(Lμ₂/(ρ₂Pₒ₂)^{0.5})^{0.5}

Here μ₂ is the dynamic viscosity coefficient of the second fluid 10, ρ₂ is its density, and Pₒ₂ is the pressure of the second fluid 10 in the upstream chamber, λ is a numerical factor, which generally is between 1 and 10. L is the length of the second fluid channel Q--P′ (FIG. 3)
L=0.5(Dₗ−Dₜ)/sin(β)

These expressions neglect the reduction in effective exit area due to the presence of first fluid in the exit orifice. Therefore, the equations provided above should be considered as approximate guides, e.g. ±30% error factors or less.

The purpose of providing all of these geometrical constraints is to make an efficient utilization of the momentum of second fluid for the purpose of atomizing first fluid, and ultimately making efficient use of the energy consumed. This purpose would be partially defeated by the presence of porous materials located inside channel 13, in which it is well known that the mechanical energy (enthalpy) in the second fluid converts into heat (internal energy of the system). Therefore, channel 13 may include, but preferably will not include, such porous structures, or other materials that may incur significant energy loss.

(c) Focusing of the First Fluid:

In the presence of second fluid flow, the first fluid 9 exiting the first fluid supply means 3 gets funnel-shaped into a jet that generally gets thinner as it flows downstream. The jet can have a variety of different configurations, e.g. a circular cross-section, or a flat planar one such as a fluid sheet for example. Any configuration can be used which provides flows through the center of the exit orifice 7, and can become much thinner as it enters the exit orifice 7 than it is at the exit 6 of the supply means 3. The forces responsible for the shaping of the first fluid 9 are believed to arise from two sources: a) the pressure gradients that set within the second fluid 10 as it flows out of channel 13 and around the exit orifice 7; and b) shear stresses that are transferred from the faster moving second fluid to the slower moving first fluid. When the source of the forces is pressure gradients alone, for example, in axi-symmetric configurations, a round first fluid jet is expected to attain a diameter dⱼ determined by the ½ power law with liquid flow rate Q (in volume per unit of time, e.g. cubic meter per second; Gañán-Calvo A. M., 1998):
dⱼ˜(8ρₗ/(π²ΔP_{g}))^{1/4}Q^{1/2}

ρₗ is the first fluid density, π is pi, and ΔP_{g} is the pressure drop in the second fluid between the upstream value (taken at the supply means exit 16) and the value at the point where dⱼ is measured (for example, at the pressure chamber exit 18, or at a point inside the pressure chamber discharge opening 15, or outside the nozzle) and ˜means approximately equal to with about a ±10% or less error margin. This equation will be herein referred to as the “flow-focusing” formula and only applies for a uniform velocity distribution along the first fluid jet radius. A similar equation exists for other geometries. The presence of shear stresses will in general, cause the jet to accelerate more than would otherwise be under the action of pressure gradients alone, and conservation of mass demands that its width will be smaller than that predicted by such “flow-focusing” equations.

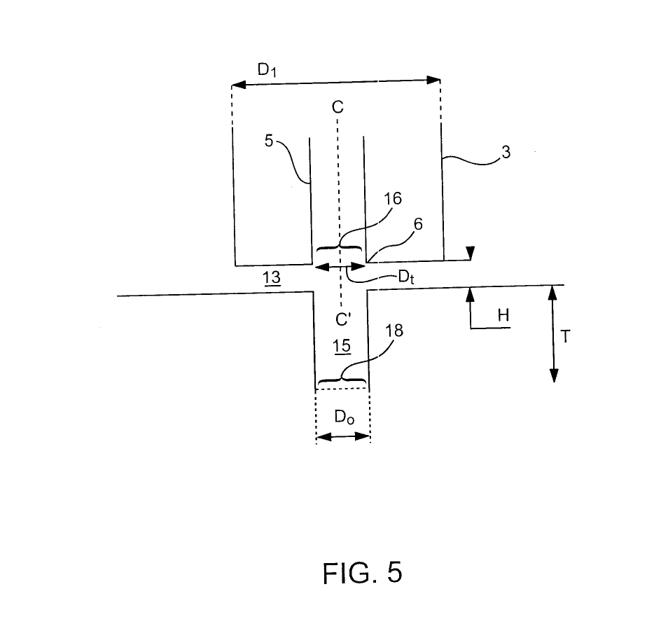

A notable consequence of the fact that the first fluid 9 is surrounded by the second fluid 10, and that all portions of the second fluid are accelerated approximately equally towards the first fluid, is that the first fluid is stabilized towards the center 14 of the pressure chamber exit orifice 18. For example, in one of the preferred device embodiments (FIG. 5), the exit 16 and the exit 18 are allowed to be of equal diameter. In all of the tests done with such embodiment the first fluid 9 was observed to flow through the center 14 of the exit orifice 18 without impacting or wetting its side walls such as at the point 7. (Due to the random nature of the particle trajectories under conditions of very high first fluid flow rates, a small degree of wetting has indeed observed, but was associated with an insignificant fraction of the first fluid 9.)

(d) Violent Focusing:

The violent focusing of the stream of fluid 9 exiting the first fluid supply means 3 is characterized by a stream of first fluid entering the exit 18 to the pressure chamber 2 which is narrower than the width of the stream of first fluid exiting the first fluid supply means 3. It is also characterized by a flow of second fluid 10 exiting the pressure chamber 2 which surrounds the first fluid everywhere, such second fluid stream having a higher speed than the first fluid stream. The violent focusing of the stream of first fluid 9 is further characterized by a rapid disintegration of such fluid over a region that spans between the exit of the first fluid supply means 3 and a nearby point in the region outside the atomizer.

(e) Gas Sonic Speeds and Shock Waves:

Sonic speeds and shock waves may take place when the second fluid is a gas. In all tests to date using such fluid choice, the pressure drop across the atomizer was such that the gas attained sonic and supersonic speeds. Under these conditions shock waves are also expected to be present.

Characteristics of supersonic flow such as shock waves may improve atomization, and may be required for optimal atomization in some cases.

Characteristics of the present invention include: (f) High frequency of droplet generation, (g) Low requirements on liquid pressure, (h) Low sensitivity of drop size to first fluid flow rate, (i) Little apparent effect of atomizer size on droplet size. These characteristics are described further below.

(f) High Frequency of Droplet Generation:

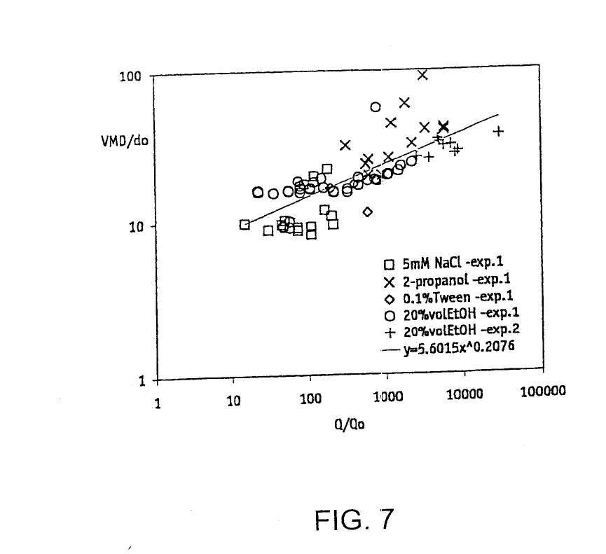

When the second fluid 10 is a gas and the first fluid 9 a liquid, experimental data demonstrate that the droplets are much smaller than predicted from the spontaneous capillary breakup, such as Rayleigh breakup in axi-symmetric configurations; (Rayleigh 1882) of an first fluid column of size dⱼ equal to that predicted by the flow-focusing formula discussed earlier. Or, what is the same, for given values of the liquid properties and operational variables (such as flow rates and pressures), the final size of the droplets is many times smaller than such flow-focusing diameter dⱼ. As a result, the frequency of droplet production is much higher than predicted by spontaneous capillary breakup of the focused jet. Accordingly, particles formed via the method described here are substantially smaller (e.g. ½ the size or less or 1/20 the size or less) than would be obtained due to spontaneous capillary break-up of the stream exiting the chamber 2 at the exit 18. (See Graph of FIG. 7)

(g) Low Requirements on Liquid Pressure:

The first fluid 9 does not have to be pushed out of its supply means 3 with a sufficiently high pressure capable of maintaining a stable liquid jet outside the tube exit 6 in the absence of second fluid flow or pressure chamber. In other words, it does not need to be pushed under pressures exceeding the so-called jetting pressure. A pre-existent first fluid jet structure coming directly out of the exit opening 6 is not required because, a explained above in (c), the first fluid meniscus is focused by the action of the second fluid pressure forces, and is thus drawn out into a continuous stream by the accelerating forces of the second fluid (pressure gradients and shear stresses).

(h) Low Sensitivity of Drop Size to First Fluid Flow Rate:

In the cases tested thus far, a low sensitivity of droplet size on flow rate has been observed. The dependence is close to a power law with exponent ⅕ of the liquid flow rate.

(i) Small Apparent Effect of Atomizer Size:

Based on the experimental data available, the drop size dependence with first fluid flow rate, second fluid pressure, and first fluid mechanical properties does not appear to involve variables characterizing the size of the atomizer. (See the below EXAMPLES.) However, under certain conditions of operation, for example at high flow rates that lead to a large fraction of the exit orifice occupied by the liquid, one would expect a certain dependence.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade.

### Examples 1-5

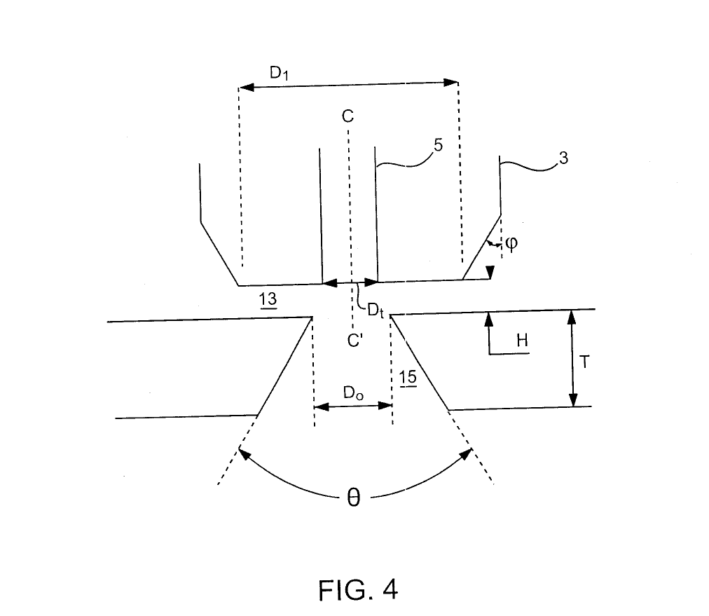

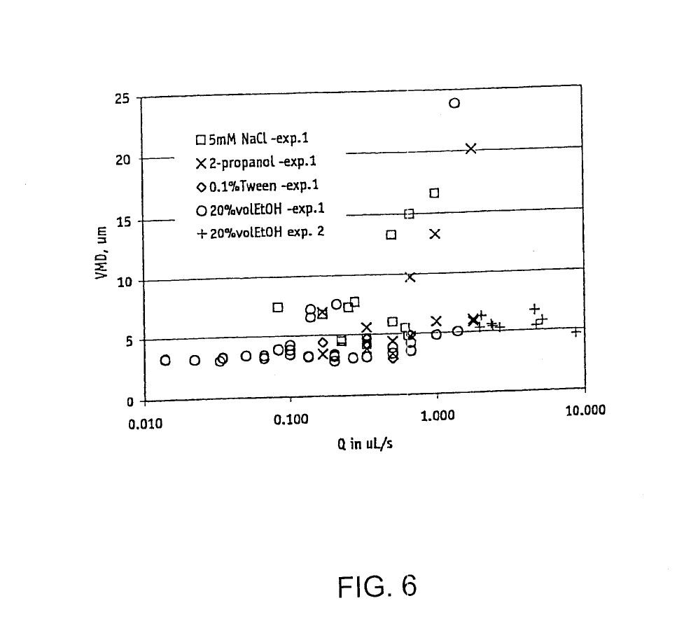

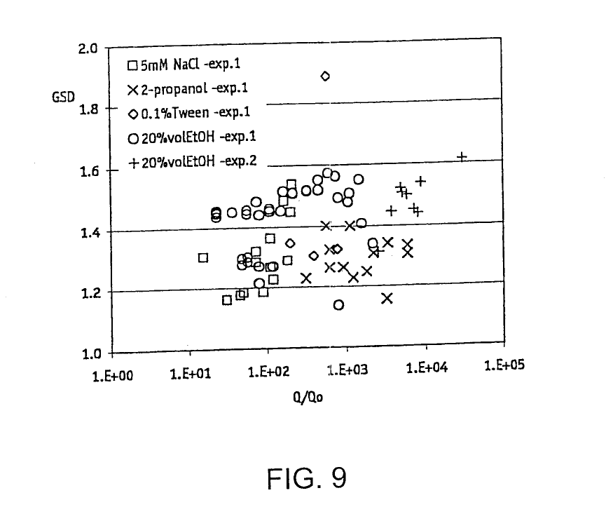

FIGS. 6-12 show results for aerosols produced by methods of the present invention using dry air and dry nitrogen as second fluids 10, and a range of liquids as first fluids 9: distilled water. 2-propanol, 20% (v/v) by volume of ethanol in water (“20% EtOH”), and 0.1% weight in volume (w/v) Polysorbate-20 in distilled de-ionized water (“0.1% Tween”). Tests were performed in four separate experiments with different atomizers. The atomizers were of an axi-symmetric type and had dimensions as specified below in Table A for variables defined in FIGS. 4 and 5. Specifically, the pressure chamber discharge opening was conveniently created by drilling a straight-through hole through a plate of thickness T.

In experiments 1-4, the droplet size was determined by phase Doppler anemometry (Lefebvre 1989; Bayvel and Orzechowski 1993) along the axis of the aerosol plume a few centimeters downstream from the exit of the atomizer. This measurement technique led to notoriously low rates of validated counts. i.e. low rates of detected light pulses (“bursts”). This problem appears to result from a combination of high droplet concentrations and high velocities. Validation count rates lower than 50% have been excluded from the sets of data presented here. As a consequence, all of the droplet size measurements in experiments 3 and 4 with were excluded from the graphs. Nevertheless, atomizer dimensions have been included in table A to indicate that stable aerosols were obtained in a third and fourth experiment with an atomizer of similar characteristics as in experiment 2, but otherwise of a very different design.

TABLE A
Atomizer geometric dimensions (in micrometers unless indicated) used in
the experiments (refer to figure for key); typical tolerance +/−15%;
(α = 0 degrees +/− 5 degrees; β = 90 degrees +/− 5
degrees) (Refer to FIGS. 4 and 5 for meaning of symbols.)

| | | | | | | | θ, |
|---|---|---|---|---|---|---|---|
| Experiment | Dₒ | Dₜ | Dₗ | H | T | Φ, degrees | degrees |
| 1 | 62 | 50 | 90 | 19 | 50 | 13 +/− 7 | 60 |
| 2 | 200 | 200 | 400 | 35 | 75 | 0 | 0 |
| 3 | 200 | 200 | 400 | 50 | 75 | 0 | 0 |
| 4 | 200 | 200 | 400 | 50-80 | 75 | 0 | 0 |
| 5 | 100 | 100 | 410 | 10-135 | 75 | 0 | 0 |
| 6 | 150 | 150 | 410 | 13.5-160 | 75 | 0 | 0 |

FIG. 6 is a graph of the volume median diameter (VMD) versus the liquid supply flow rate for four different liquids.

In FIG. 7 the volume median diameter and liquid flow rates have been non-dimensionalized using similar variables to those identified in the flow-focusing literature (Gañán-Calvo 1998), dₒ and Qₒ:
dₒ=σ/ΔP_{g}
and
Qₒ=(σ⁴(ρₗΔP_{g}³)^{1/2}

where σ is the interfacial tension of the liquid-gas interface (Newton/meter). The definition of the pressure drop ΔP_{g} is based on the upstream (stagnation) value Pₒ, estimated to be a fair representation of the pressure at the exit of first fluid supply means 6 (FIG. 5), and the value P* at the sonic point, expected to be located at exit 18 of the pressure chamber 3. The sonic pressure P* was computed using the well-known isentropic expression:
P*=Pₒ(2/(k+1))^{k/(k-1)}

where k is the heat capacity ratio of the gas (equal to 1.4 for dry air and dry nitrogen; White 1994). Therefore
ΔP_{g}=Pₒ−P*=Pₒ(1−(2/(k+1))^{k(k-1)})

Thus, for both dry air and dry nitrogen,
ΔP_{g}=0.4717Pₒ

In experiments 1 through 4, Pₒ was varied between 200 kPa and 700 kPa.

The best power law fit to the available data (FIG. 7) is:
VMD/dₒ=5.60(Q/Qₒ)^{0.208}

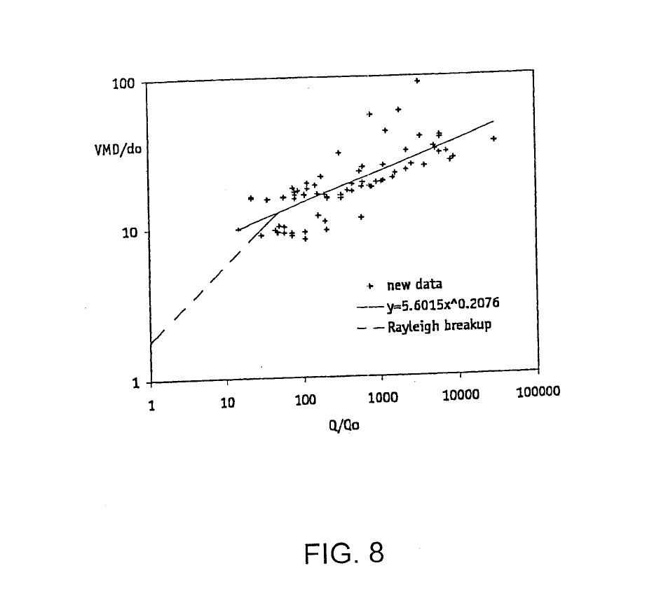

FIG. 8 graphs the new fit characteristic of the new method together with the one which would correspond to the Rayleigh breakup of a flow-focused jet at the same conditions of liquid properties, flow rate, and gas pressure (thus equal dₒ, Q, and Qₒ in each case). The results shown in FIG. 8 are based on the theoretical assumption that Rayleigh breakup of a flow-focused jet would result in droplets of uniform diameter (VMD) equal to 1.89 times the jet diameter (Brodkey 1995), and on applying the flow-focusing equation for the jet diameter given earlier, to estimate VMD as follows:
VMD=1.89(8ρₗ/(π²ΔP_{g}))^{1/4}Q^{1/2}

This expression has been cast into dimensionless form using the definitions of dₒ and Qₒ:
VMD/dₒ=1.89(8/π²)^{1/4}(Q/Qₒ)^{1/2}

In FIG. 8 the “Rayleigh breakup” line, based on this expression, has been graphed between the limits believed to occur in reality. If this expression could be extrapolated to higher Q/Qo values, it would predict larger drop sizes at equal conditions of Q/Qo and do. But, more importantly, because the dependence with Q/Qo is much less pronounced than for flow-focused jets, the range of liquid flow rates over which a certain band of desired drop sizes can be generated is much wider than from Rayleigh breakup of flow-focused jets. These conclusions should apply as well when a comparison is being made to non-Rayleigh breakup of flow-focused jets, provided the droplet diameters become similar to the jet diameter.

Another notable result is that data from dissimilar atomizers seems to follow the same scaling law. In other words, based on currently available data, the scaling law (at least its exponent of approximately ⅕) appears to be relatively insensitive to the scale of the atomizer. However, in general, differences from this behavior may be encountered, when practicing the methods disclosed.

The proposed atomization system obviously requires delivery of the first fluid 9 to be atomized and the second fluid 10 to be used in the resulting suspension of particles. Both fluids should be fed at a rate ensuring that the system lies within a desired parameter window. For example, not exceeding a certain ratio of second to first fluid mass flow rates is generally an important consideration. Multiplexing a number of atomizers is effective when the total amount of first fluid flow-rate needed exceeds that obtained from an individual atomizer or cell. More specifically, a plurality of feeding sources 3 or holes therein forming tubes in the first fluid supply means 3 may be used to increase the overall rate at which particle suspensions are created. The flow-rates used should also ensure the mass ratio between the flows is compatible with the specifications of each application.

The second fluid and first fluid can be dispensed by any type of continuous delivery system (e.g. a compressor or a pressurized tank the former and a volumetric pump or a pressurized bottle the latter). If multiplexing of atomizers is needed, the first fluid flow-rate should be as uniform as possible among cells; this may entail propulsion through several capillary needles, porous media or any other medium capable of distributing a uniform flow among different feeding points.

Although a single first fluid supply means 3 is shown in FIGS. 1-5, it is, of course, possible to produce a device with a plurality of feeding members 3 where each feeding member feeds fluid to an array of outlet orifices 18 in a single surrounding pressure chamber 2. These feeding members can be separate solid bodies, or can share one or more solid components. For example, a row of feeding channels to supply first fluid 9 can be created by joining two halves, each patterned with a series of half channels needed to supply the first fluid. In addition, the first fluid supply means may be planar with grooves therein, but need not be strictly planar, and may be a curved feeding device comprised of two surfaces that maintain approximately the same spatial distance between the two pieces of the first fluid supply means. Such curved devices may have any level of curvature, e.g. circular, semicircular, elliptical, hemi-elliptical, etc.

### Example 6

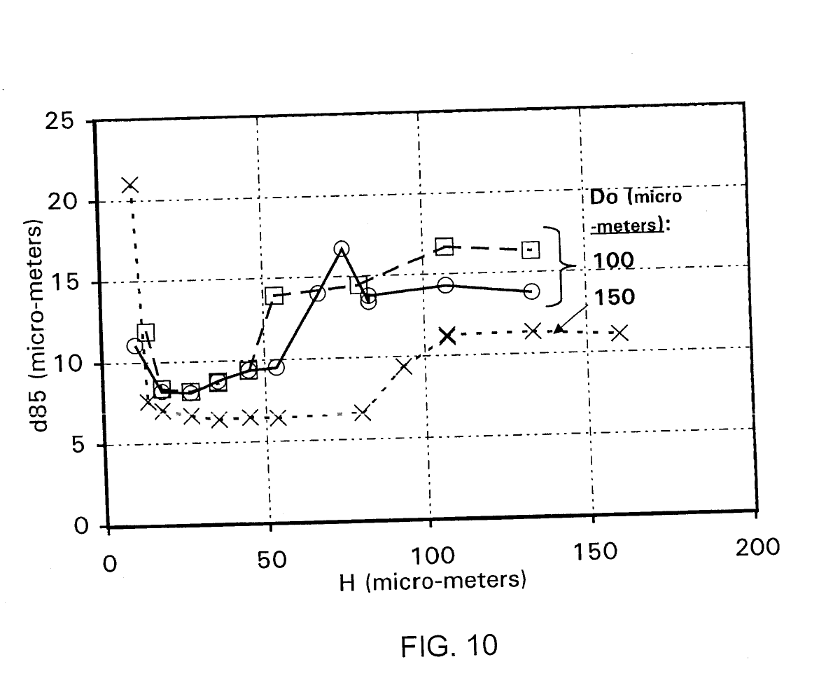

FIGS. 10, 11, and 12 report results from a separate experiment in which the aerosol size distribution was carefully measured as a function of the distance between the first fluid supply means and the pressure chamber, H. Aerosol size distributions were measured outside the atomizer using a standard aerosol measurement technique called laser diffraction (using a Sympatec HELOS system). A device was designed having a configuration as that shown on FIG. 5. The geometric parameters for this system are recorded in the last line of TABLE A above. Measurements of the particle size distribution were made with de-ionized water as first fluid and dry nitrogen as second fluid, at a water flow rate of 35 ml/hr and a pressure in the pressure chamber measured upstream from channel 13 relative to the room into which the aerosol was discharged, of 10 bar. Presented are two statistics that define the particle size: d85 and GSD. d85 represents the diameter under which is represented 85% of the volume of the aerosol measured. (For example, using this nomenclature, the volume median diameter VMD described earlier would be expressed as d50.) GSD is a measure of the width of the distribution in droplet sizes, and is equal to the so-called geometric standard deviation.

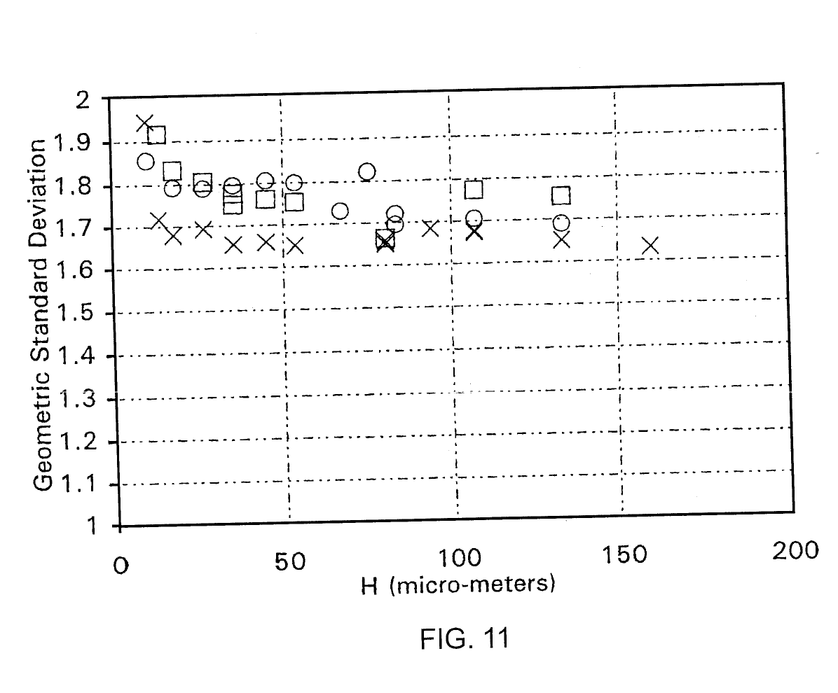

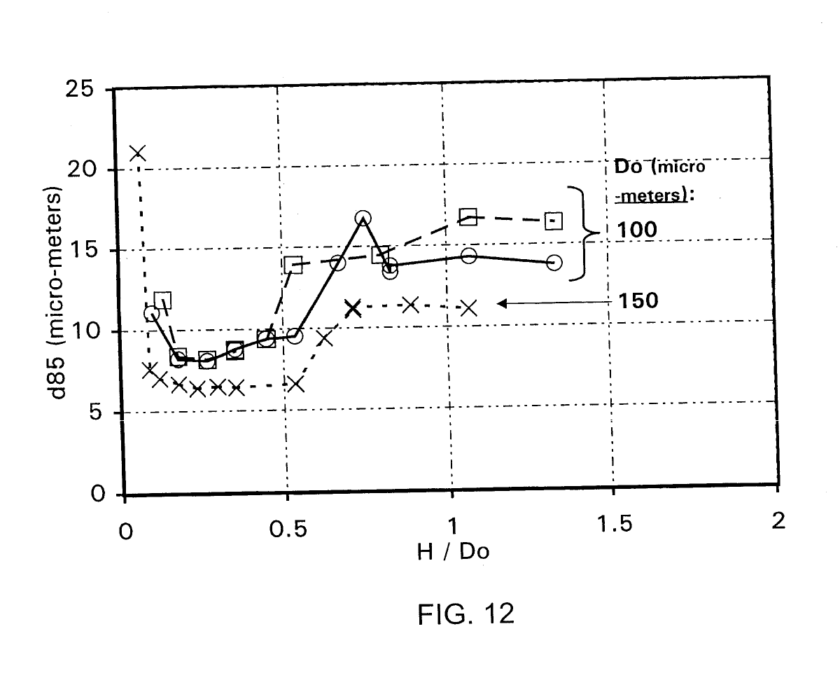

FIG. 10 graphs d85 as a function of the channel with H. It can be seen that d85 is large at the largest values of H. but it is quite small at smaller values of H. and then rises again for even lower values of H. The transition seen at intermediate values of H represents a transition from a certain mode of atomization to another, more efficient violent focusing mode. All tests represent conditions of approximately constant second fluid flow rate (given that the pressure upstream, and the geometry of the pressure chamber exit where the sonic condition determining the mass flow rate is assumed to exist are kept constant.) Interestingly, the width of the distribution is not worsened when this transition takes place. In fact, FIG. 11 shows that the GSD stays nearly constant throughout the entire range of conditions tested. FIG. 12 shows d85 versus the ratio of H to the inner diameter of the liquid supply channel (Dₜ).

### Drug Delivery Device

A device of the invention may be used to provide particles for drug delivery, e.g. the pulmonary delivery of aerosolized pharmaceutical compositions comprised of a drug alone or with a pharmaceutically acceptable carrier. The device would produce aerosolized particles of a pharmaceutically active drug for delivery to a patient by inhalation. The device is comprised of a first fluid feeding source such as a channel to which formulation is added at one end and expelled through an exit opening. The feeding channel is surrounded by a pressurized chamber into which second fluid is fed and out of which second fluid is expelled from an opening. The opening from which the second fluid is expelled is positioned directly in front of the flow path of first fluid expelled from the feeding channel. Various parameters are adjusted so that pressurized second fluid surrounds first fluid flowing out of the feeding channel in a manner so as to reduce the dimension of the flow which is then broken up on leaving the chamber. The aerosolized particles are inhaled into a patient's lungs and thereafter reach the patient's circulatory system. Examples of the second fluid used are air, nitrogen, carbon dioxide, etc., and mixtures thereof. Examples of first fluid are a drug dissolved or suspended in an aqueous formulation, ethanolic formulation, etc., and mixtures thereof.

### Production of Dry Particles

The method of the invention is also applicable in the mass production of dry particles. Such particles are useful in providing highly dispersible dry pharmaceutical particles containing a drug suitable for a drug delivery system, e.g. implants, injectables or pulmonary delivery. The particles formed of pharmaceutical are particularly useful in a dry powder inhaler due to the small size of the particles (e.g. 1-5 micro-meters in aerodynamic diameter) and conformity of size (e.g. ±3% to ±30% difference in diameter) from particle to particle. Such particles should improve dosage by providing accurate and precise amounts of dispersible particles to a patient in need of treatment. Dry particles are also useful because they may serve as a particle size standard in numerous applications.

For the formation of dry particles, the first fluid is preferably a liquid, and the second fluid is preferably a gas, although two liquids may also be used provided they are generally immiscible. Atomized particles are produced within a desired size range (e.g., 1 micron to about 5 micro-meters). The first fluid is preferably a solution containing a volatile solvent and a high concentration of solute drug. Alternatively, the first fluid is a suspension containing a uniform concentration of suspended matter. In either case, the liquid solvent quickly evaporates upon atomization (due to the small size of the particles formed) to leave very small dry particles.

### Fuel Injection Apparatus

The device of the invention is useful to introduce fuel into internal combustion engines by functioning as a fuel injection nozzle, which introduces a fine spray of aerosolized fuel into the combustion chamber of the engine. The fuel injection nozzle has a unique fuel delivery system with a pressure chamber and a fuel source. Atomized fuel particles within a desired size range (e.g., 5 micron to about 500 micro-meters, and preferably between 10 and 100 micro-meters) are produced from a liquid fuel formulation provided via a fuel supply opening. Different size particles of fuel may be required for different engines. The fuel may be provided in any desired manner, e.g., forced through a channel of a feeding needle and expelled out of an exit opening of the needle. Simultaneously, a second fluid, e.g. air, contained in a pressure chamber which surrounds at least the area where the formulation is provided (e.g., surrounds the exit opening of the needle) is forced out of an opening positioned in front of the flow path of the provided fuel (e.g. in front of the fuel expelled from the feeding needle). Various parameters are adjusted to obtain a fuel-fluid interface and an aerosol of the fuel, which allow formation of atomized fuel particles on exiting the opening of the pressurized chamber.

Fuel injectors of the invention have two significant advantages over prior injectors. First, fuel generally does not contact the periphery of the exit orifice from which it is emitted because the fuel stream is surrounded by an oxidizing gas (e.g. air or oxygen) which flows into the exit orifice. Thus, clogging of the orifice is eliminated or substantially reduced. In addition, formation of carbon deposits around the orifice exit is also substantially reduced or eliminated. Second, the fuel exits the orifice and forms very small particles which may be substantially uniform in size, thereby allowing faster and more controlled combustion of the fuel.

### Microfabrication

Molecular assembly presents a ‘bottom-up’ approach to the fabrication of objects specified with incredible precision. Molecular assembly includes construction of objects using tiny assembly components, which can be arranged using techniques such as microscopy, e.g. scanning electron microspray. Molecular self-assembly is a related strategy in chemical synthesis, with the potential of generating non-biological structures with dimensions as small as 1 to 100 nanometers, and having molecular weights of 104 to 10¹⁰ daltons. Microelectro-deposition and microetching can also be used in microfabrication of objects having distinct, patterned surfaces.

Atomized particles within a desired size range (e.g., 0.001 micron to about 0.5 micro-meters) can be produced to serve as assembly components to serve as building blocks for the microfabrication of objects, or may serve as templates for the self-assembly of monolayers for microassembly of objects. In addition, the method of the invention can employ an atomizate to etch configurations and/or patterns onto the surface of an object by removing a selected portion of the surface.

### REFERENCES

1. Schuster, J. A., Rubsamen, R. M., Lloyd, P. M. and Lloyd, L. J. (1997) “The AERx Aerosol Delivery System.” Pharmaceutical Research vol 14 (3) pp 354-357
2. A. M. Ganan-Calvo (1998) “Generation of Steady Liquid Microthreads and Micron-Sized Monodisperse Sprays in Gas Streams.” Physical Review Letters, vol 80 (2) pp 285-288
3. U.S. Pat. No. 6,119,953, “A Liquid Atomization Procedure”. A. Ganan Calvo, A. Barrero Ripoll
4. Bayvel L. and Z. Orzechowski, (1993) Liquid Atomization. Taylor and Francis. Washington D.C.
5. Layernia E. J. and Y. Wu, (1996) Spray Atomization and Deposition. John Wiley & Sons Ltd. Chichester. West Sussex. England.
6. Lefebvre A. H. (1989) Atomization and Sprays. Hemisphere Publ. Co., New York.
7. Gretzinger-Marshall, (1961) “Characteristics of Pneumatic Atomization” A.I.Ch.E. Journal, June 1961, pages 312-318.

The instant invention is shown and described herein in a manner which is considered to be the most practical and preferred embodiments. It is recognized, however, that departures may be made therefrom which are within the scope of the invention and that obvious modifications will occur to one skilled in the art upon reading this disclosure.

1.-26. (canceled)

27. A method of generating an aerosol, comprising the steps of:
forcing a liquid formulation comprised of a pharmaceutically active drug through a feeding supply channel and out of an exit opening;
filling a pressure chamber with a gas which chamber is in fluid connection with the exit opening of the feeding supply channel;
forcing the gas toward and into the liquid in a manner which destabilizes the liquid into particles having a diameter less than the diameter of the exit opening of the feeding supply channel; and
allowing the gas to exert force on the liquid and force particles of the liquid out of an exit orifice of the pressure chamber positioned downstream of a direction of flow of the liquid stream.

28. The method of claim 27, wherein the particles formed comprise particles of 1-5 micrometers in aerodynamic diameter.

29. The method of claim 28, further comprising:
inhaling the particles into a patient's lungs.

30. The method of claim 29, wherein liquid of the particles evaporates leaving dry particles to be inhaled.

31. The method of claim 27, wherein the particles formed comprise particles of 1-3 micrometers in diameter.

32. The method of claim 27, wherein the gas is forced into the liquid at an angle in a range of from about 15° C. to about 90° to the liquid flow direction.

33. The method of claim 28, wherein the gas is forced into the liquid at an angle in a range of from about 45° C. to about 90° to the liquid flow direction.

34. The method of claim 29, wherein the gas is forced into the first liquid stream circumference at an angle in a range of from about 90±5° to the liquid flow direction.

35. The method of claim 27, wherein the energy source causes the gas to converge toward the liquid along streamlines that form an angle of 45° or greater with respect to the liquid.

36. The method of claim 27, wherein the exit opening of the feeding supply channel has a diameter in the range of about 5 to about 10,000 microns.

37. The method of claim 29, wherein the exit opening of the feeding supply channel is positioned at a distance in a range of from about 5 to about 10,000 microns from the exit orifice of the pressure chamber.

38. The method of claim 28, wherein the exit opening of the pressure chamber has a diameter in the range of about 5 to about 10,000 micro-meters.

39. The method of claim 40, wherein the ratio of the exit opening of the feeding supply channel width to the pressure chamber exit orifice width is greater than about 0.5.

40. The method of claim 40, wherein the ratio of the exit opening of the feeding supply channel width to the pressure chamber exit orifice width is in the range of about 0.7 to about 1.2.

41. The method of claim 38, wherein the ratio of the exit opening of the feeding supply channel width to the pressure chamber exit orifice width is in the range of about 0.8 to about 1.0.

42. The method of claim 38, wherein the ratio of the separation between the exit opening of the feeding supply channel and the pressure chamber exit orifice, to the pressure chamber exit orifice width, is less than about 1.5.

43. The method of claim 38, wherein the ratio of the separation between the exit opening of the feeding supply channel and the pressure chamber exit orifice, to the pressure chamber exit orifice width, is in the range of is less than about 1.0.

44. The method of claim 38, wherein the ratio of the separation between the exit opening of the feeding supply channel and the pressure chamber exit orifice, to the pressure chamber exit orifice width is in the range of is in the range of about 0.2 to about 0.7.

45. The method of claim 38, wherein the exit opening of the feeding supply channel and the pressure chamber exit orifice are substantially circular.

46. The method of claim 38, wherein the gas is forced into the liquid by being forced through a channel, one wall of the channel having an angle to the centerline of the feeding supply channel in the range of about 75 to about 105 degrees.

47. The method of claim 38, wherein the exit orifice of the pressure chamber has a diameter in the range of about 15 to about 400 micro-meters.

48. The method of claim 38, wherein the exit opening of the feeding supply channel has a diameter in the range of about 15 to about 300 micrometers.

49. The method of claim 38, wherein the exit opening of the container is positioned at a distance in a range of from about 15 to about 300 micro-meters from the orifice in the pressure chamber.

50. The method of claim 38, wherein the gas is selected from the group consisting of air, nitrogen, carbon dioxide, helium, argon and mixtures thereof.

51. The method of claim 49, wherein the gas is comprised of carbon dioxide.
